(19) United States
(12) Patent Application Publication
(10) Pub. No.: US 2008/0193545 A1
(43) Pub. Date: Aug. 14, 2008

(54) Use of Glycerol Dipalmitostearate for Improving the Bioavailability of Protein Active Ingredients in Subcutaneous or Intramuscular Injectable Formulations

(75) Inventors: Joel Richard, Saint Barthelemy D'Anjou (FR); Frantz Deschamps, Nancy (FR); Anne-Marie De Conti, Saint Maur Des Fosses (FR); Olivier Thomas, Angers (FR); Richard Aubreton, Angers (FR)

Correspondence Address: FOLEY AND LARDNER LLP;SUITE 500, 3000 K STREET NW, WASHINGTON, DC, 20007, US

(73) Assignee: ETHYPHARM, Houdan (FR)

(21) Appl. No.: 11/547,754

(22) PCT Filed: Apr. 7, 2005
(86) PCT No.: PCT/FR05/00849

§ 371 Date: Sep. 5, 2007

(30) Foreign Application Priority Data
Apr. 7, 2004 (FR) 0403639

(51) Int. Cl. A61K 9/50; A61K 38/21

(52) U.S. Cl. 424/498; 424/85.7; 424/499; 424/490

(57) ABSTRACT

The invention relates to the use of lipids as formulation agents for increasing the bioavailability of protein active ingredients in subcutaneous or intramuscular injectable formulations.

The present invention relates to a method for increasing the bioavailability of protein active ingredients in subcutaneous or intramuscular injectable formulations.

Recent advances in the field of biotechnologies, and more particularly in genetic engineering, have led to the development of numerous protein active ingredients, defined as peptides and proteins, notably for use in the treatment of serious pathologies (cancer, anemia, multiple sclerosis, hepatitis, etc.). However, for these macromolecular active ingredients, which are fragile and of very low bioavailability, the development of formulations that are stable, efficient and well-tolerated by the patient remains a true challenge. In particular, because of their weak stability in the gastrointestinal tract (degradation at low pH and proteolysis) and their weak permeability across the intestinal barrier, protein active ingredients cannot be administered by oral route. In this regard, although an improvement in the permeability of peptides and proteins across the epithelial membrane is conceivable, oral bioavailability remains less than a few percent nevertheless, which prevents the development of oral forms of these active ingredients. Thus, in this context, administration by injectable or parenteral route remains the preferred administration route for protein active ingredients. Therefore, the development of novel injectable dosage forms or injection devices is more likely to achieve therapeutic and commercial success than the use of a novel administration route, on the condition, however, that the injections provide the desired safety and effectiveness. A review of the state of the art of the novel dosage forms of products arising from biotechnologies was published recently by G. Orive et al. (“Drug delivery in biotechnology: present and future”; Current Opinion in Biotechnology 14, 2003, 659-664).

Numerous formulations of peptides and proteins that are injectable by intravenous, intramuscular or subcutaneous route are already marketed or are under development. A review of the formulations of protein active ingredients available commercially or under development was published recently by J. L. Cleland et al. (“Emerging protein delivery methods”; Current Opinion in Biotechnology 12, 2001, 212-219). As these authors show, these formulations still require significant improvements related to several problems which remain unresolved. First, proper observance of the treatment regimen is a major concern in the treatment of the elderly and the young, for whom repeated parenteral injections are poorly tolerated. Moreover, frequent side effects directly due to the injection and to peak plasma concentration, such as flu-like symptoms, anorexia, weight loss, digestive problems, etc., are also observed. Lastly, the high systemic concentrations required for these protein active ingredients to produce the desired effect on the targeted tissues or organs are very often responsible for a systemic toxicity (hematological, etc.) that is harmful and detrimental to the patient; these elevated concentrations, which are required for the therapeutic window, are obtained by the frequent injection of high doses. The first two problems were solved partially by polymer-based protein active ingredient prolonged delivery systems (implants, microspheres, gels), although these systems have not solved the problems of loss of activity (denaturation) and of aggregation and/or precipitation related to the methods and formulation excipients used (for example, see the article by M. van de Weert et al.: “Protein instability in poly(lactic-co-glycolic acid) microparticles”; Pharm. Res. 17, 2000, 1159-1167). On the other hand, the third point, which relates to the exposure of the patient to the protein active ingredient, remains unresolved today, and in many cases leads to the patient's refusal to follow the treatment. This problem is related to the weak bioavailability of protein active ingredients, and more particularly to proteins administered by subcutaneous or intramuscular route, which leads to the use of high therapeutic doses. After subcutaneous (SC) administration, absolute bioavailability (versus intravenous administration at the same dose) is commonly in the range between 12% and 70% for proteins with a molar mass greater than or equal to 20,000 g/mol (erythropoietin, interferon beta, interferon gamma, etc.); it can be slightly higher, reaching approximately 80% for proteins of lower molar mass. Likewise, the absolute bioavailability of some cytokines, such as interferon beta, is very low after intramuscular (IM) administration, commonly less than or equal to 10% (for example, see the article by V. Bocci: “Physicochemical and biologic properties of interferons and their potential uses in drug delivery systems”; Critical Reviews in Therapeutic Carrier Systems 9 (2), 1992, 91-133). This problem of bioavailability after subcutaneous administration also persists for novel forms of bioconjugated proteins with prolonged systemic circulation times, such as PEGylated proteins (i.e., on which water-soluble polymer groups such as poly(ethylene glycol) or (PEG) are bound covalently), or glycosylated proteins (i.e., on which water-soluble polymer groups such as mono- or poly-saccharides are bound covalently), as was shown in 2003 in the review by P. Caliceti et al. (“Pharmacokinetic and biodistribution properties of poly(ethylene glycol)-protein conjugates”; Advanced Drug Delivery Reviews 55, 2003, 1261-1277).

The exact origin of the weak absolute bioavailability following SC or IM injection has not been explained, but in the literature it is attributed to possible degradation (proteolysis) or metabolization of the protein active ingredient at the injection site or during lymphatic transport to the plasma. The degradation of cytokines by proteases after SC administration thus reduces their bioavailability by more than 30% (for example, see the article by B. Rouveix: “Clinical pharmacology of cytokines”; Eur. Cytokine Network 8 (3), 1997, 291-293). A review of the state of knowledge on the absorption of proteins and the mechanisms of transport towards the circulatory system after administration by SC injection was published recently by C. J. H. Porter et al. (“Lymphatic transport of proteins after subcutaneous administration”; J. Pharm. Sci. 89 (3), 2000, 297-310).

Lastly, it should be noted that if the bioavailability of proteins injected by SC or IM route is low (<20%), this generates a significant loss of the protein of which only a small portion will be of use therapeutically, which leads to a significant increase in the cost of producing the drug for the pharmaceutical manufacturer (J. L. Cleland et al., already cited).

In this context, the applicant has discovered that the formulation of protein active ingredients with lipid compounds used as formulation agents makes it possible, in an unexpected way, to increase the absolute bioavailability of these active ingredients after administration by the subcutaneous or intramuscular injection route. This increase in bioavailability makes it possible to respond to the problem of therapeutic dose and associated toxicity, as well as to the economic problem posed by the production of drugs containing these active ingredients.

Also, the object of the present invention is to provide a method for improving the bioavailability of protein active ingredients in injectable subcutaneous or intramuscular formulations.

According to the present invention, it has been demonstrated that the use of lipids as formulation agents has made it possible to increase the bioavailability of protein active ingredients in subcutaneous or intramuscular injectable formulations.

As a consequence, the object of the present invention relates to the use of lipids as formulation agents to increase the bioavailability of protein active ingredients in subcutaneous or intramuscular injectable formulations.

According to the invention, the lipids are used in liquid or solid form at ambient temperature. In an advantageous embodiment, they are used in solid form.

According to another specific embodiment of the invention, the lipids are chosen from among the group comprising the phospholipids, the C₈-C₂₂ mono-, di- and tri-glyceride fatty acids, the fatty acid esters, the fatty alcohols, the glycoglycerolipids, the sucrose esters, and mixtures thereof.

According to another particularly advantageous embodiment of the invention, the phospholipids are chosen from among the group comprising phosphatidylcholine, phosphatidylglycerol, diphosphatidylglycerol, dipalmitoyl-phosphatidylcholine, dioleylphosphatidyl-ethanolamine, dioleylphosphatidylcholine and dimyristoyl-phosphatidylglycerol, and mixtures and lyso derivatives thereof.

According to another advantageous embodiment of the present invention, the mono-, di- and tri-glycerides are chosen from among the group comprising the C₈-C₁₈ mono-, di- and tri-glycerides, in particular the caprylic, capric, lauric, myristic, palmitic, or stearic acid mono-, di- and tri-glycerides and mixtures thereof.

According to a particularly advantageous embodiment of the present invention, the fatty acid esters are chosen from among the group comprising the C₈-C₂₂ fatty acid esters.

According to another advantageous embodiment of the present invention, the fatty acid esters are chosen from among the group comprising the C₁₂-C₁₈ fatty acid esters and mixtures thereof, in particular from among ethyl palmitate, ethyl myristate, isopropyl myristate, octyldodecyl myristate and mixtures thereof.

According to a highly advantageous embodiment of the invention, the solid lipid is glycerol dipalmitostearate, still more advantageously Precirol® ATO 5 (glycerol dipalmitostearate, type I atomized according to the nomenclature of the European Pharmacopeia, 3rd edition).

According to the invention, the protein active ingredient is a peptide or a peptide derivative chosen from the group comprising the somatotropin analogs, somatomedin-C, gonadotropin-releasing hormone, follicle-stimulating hormone, luteinizing hormone-releasing hormone (LHRH) and its analogues such as leuprolide, nafarelin and goserelin, LHRH agonists and antagonists, growth hormone releasing factor, calcitonin, colchicine, gonadotropins such as chorionic gonadotropin, oxytocin, octreotide, somatotropin, amino-acid associated somatotropin, vasopressin, adrenocorticotropic hormone, epidermal growth factor, prolactin, somatostatin, protein-associated somatropin, cosyntropin, lypressin, polypeptides such as thyrotropin-releasing hormone, secretin, pancreozymin, enkephalin, glucagon, and endocrine agents secreted internally and distributed by blood flow.

Cited among the other agents which can be delivered according to the invention are, in particular, alpha-1 antitrypsin, factor VIII, factor IX and other coagulation factors, insulin and other peptide hormones, growth hormone, cortical androgen-stimulating hormone, thyroid stimulating hormone and the other pituitary hormones, parathyroid hormone-related protein, interferons alpha, beta, gamma and delta, erythropoietin (EPO), growth factors such as granulocyte colony-stimulating factor (GCSF), granulocyte-macrophage colony-stimulating factor (GMCSF), nerve growth factor (NGF), neurotrophic factors such as GDNF, insulin-like growth factors, tissue plasminogen activator, CD4, dDAVP, tumor necrosis factor (TNF), pancreatic enzymes, lactase, cytokines, interleukin-1 receptor antagonists, interleukin-2, tumor necrosis factor (TNF) receptor, tumor suppressing proteins, cytotoxic proteins, etc.

The active ingredient can also be a mixture of peptides and/or proteins such as those listed above.

According to a particularly advantageous embodiment of the present invention, the protein active ingredient is a cytokine, even more advantageously an interferon, in particular interferon α-2b.

The protein active ingredients used according to the invention are preferentially contained in particles ranging between 0.1 μm and 100 μm in size, even more advantageously ranging between 1 μm and 25 μm in size. They are obtained by any method known to those skilled in the art, such as, but not limited to, lyophilization, atomization, atomization-freezing, crystallization, precipitation, grinding, and methods using a fluid at supercritical pressure.

The protein content of the particles of protein active ingredient is in the range between 0.01% and 100%, and is adjusted according to the knowledge of those skilled in the art to obtain the desired therapeutic dose. These particles of protein active ingredient can also contain commonly-used excipients and additives, such as, for example, surfactants, polymers, sugars, polysaccharides, salts, buffers, and other proteins.

The methods which can be implemented to obtain systems containing a protein active ingredient formulated with a lipid agent are numerous and are generally known to those skilled in the art. They are based, for example, on techniques of emulsification, dispersion, emulsion-extraction of solvent, hot-melt emulsion-cooling, double emulsion, oil-in-water emulsion, precipitation by tilting of solvent, coacervation (phase separation), atomization followed by cooling, prilling, coating in a fluidized bed, encapsulation using a fluid at supercritical pressure, extrusion, extrusion-spheronization, thermoforming or compression (for implants). A method of encapsulation using a fluid at supercritical pressure as a solvent or a non-solvent of the lipid or lipids of the formulation is implemented preferentially. In this case, the lipid agent used is preferably soluble in the supercritical fluid. Preferentially, the supercritical fluid consists of carbon dioxide (CO₂), alone or in the presence of co-solvents.

The formulated systems obtained have structures of the following types: nanocapsule, microcapsule (lipid-core, protein-shell structure), nanosphere, microsphere, cylindrical or discoidal implants (lipid matrix structure containing the dispersed protein active ingredient). Preferentially, they have a matrix structure.

The formulated systems lack any PLA/PLGA, acrylic, cellulose or vinyl polymers.

Their content in particles of protein principle ingredient (load factor) is in the range between 0.1% and 95%, advantageously between 0.5% and 25%, still more advantageously between 1% and 10%.

The formulated systems containing the protein active ingredient thus obtained have a mean size in the range between 1 μm and 500 μm.

For the lipid particulate formulated systems, the mean diameter is preferentially in the range between 10 μm and 100 μm; for the lipid implants, the mean diameter is preferentially in the range between 10 μm and 300 μm.

Prior to injection, the particulate systems are redispersed in a liquid carrier for subcutaneous or intramuscular injection. In a nonrestrictive way, they are aqueous solutions, and preferentially buffered, isotonic aqueous solutions of defined viscosity and adjusted by water-soluble and/or surfactant polymers, and contain surfactants making it possible to obtain a homogeneous and stable dispersion of the particles. The composition of these liquid carriers for injection is adjusted by those skilled in the art to obtain a subcutaneous or intramuscular injectable dispersion.

The method according to the invention leads to an increase in the bioavailability of the protein active ingredient and makes it possible to use smaller quantities of active ingredients, thus avoiding the treatment stoppages related to the high toxicity of the aforesaid protein active ingredients.

The following examples, tables and figures illustrate the invention.

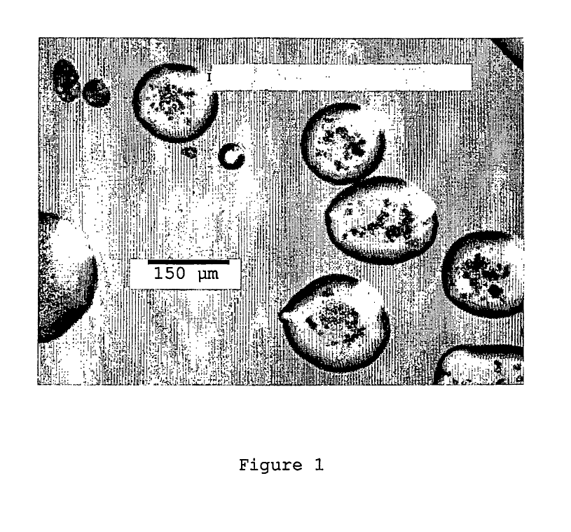

FIG. 1 is an optical microscope photograph of microparticles Ml after the melting of the lipid as described in example 1.

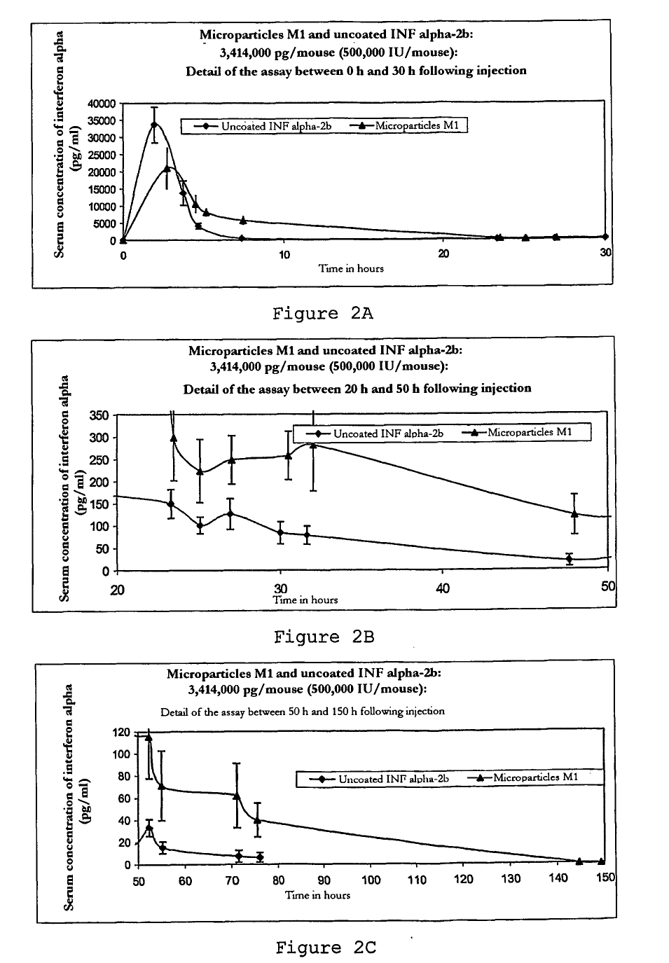

FIG. 2 represents the serum concentration of interferon α in the mouse for a dose of interferon α-2b of 500,000 IU/mouse: (A) 0 h to 30 h after the injection of uncoated INFα-2b or of microparticles M1 prepared according to example 1, (B) 20 h to 50 h after the injection of coated INFα-2b or of microparticles M1 prepared according to example 1, (C) 50 h to 150 h after the injection of uncoated INFα-2b or of microparticles M1 prepared according to example 1.

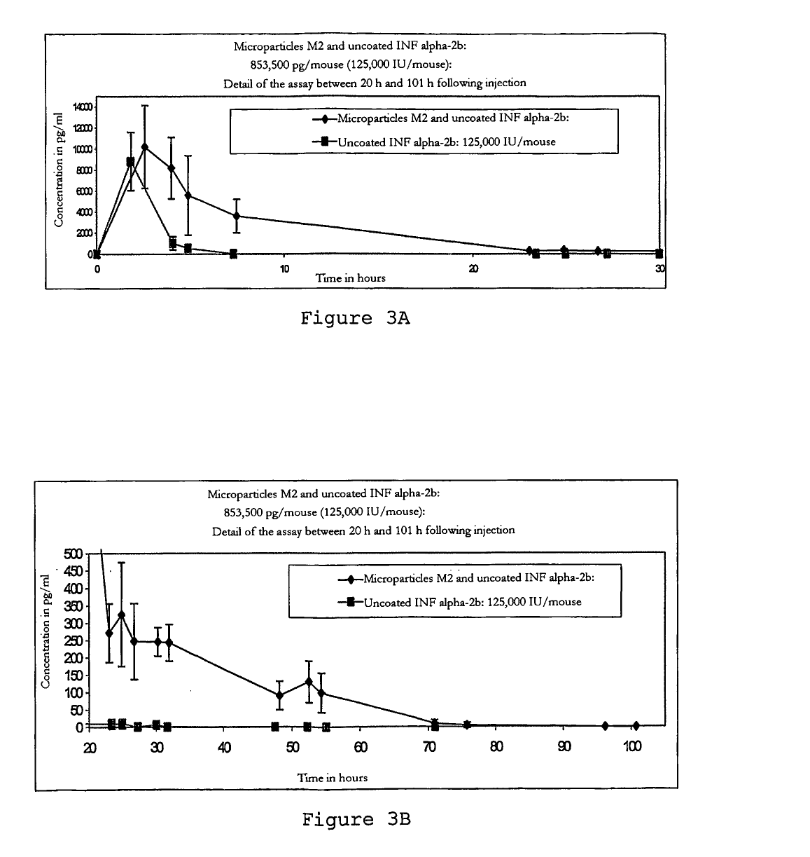

FIG. 3 represents the serum concentration of interferon a in the mouse for a dose of interferon α-2b of 125,000 IU/mouse: (A) 0 h to 30 h after the injection of uncoated INFα-2b or of microparticles M2 prepared according to example 2 and suspended in the medium from example 4, (B) 20 h to 101 h after the injection of uncoated INFα-2b or of microparticles M2 prepared according to example 2 and suspended in the medium from example 4.

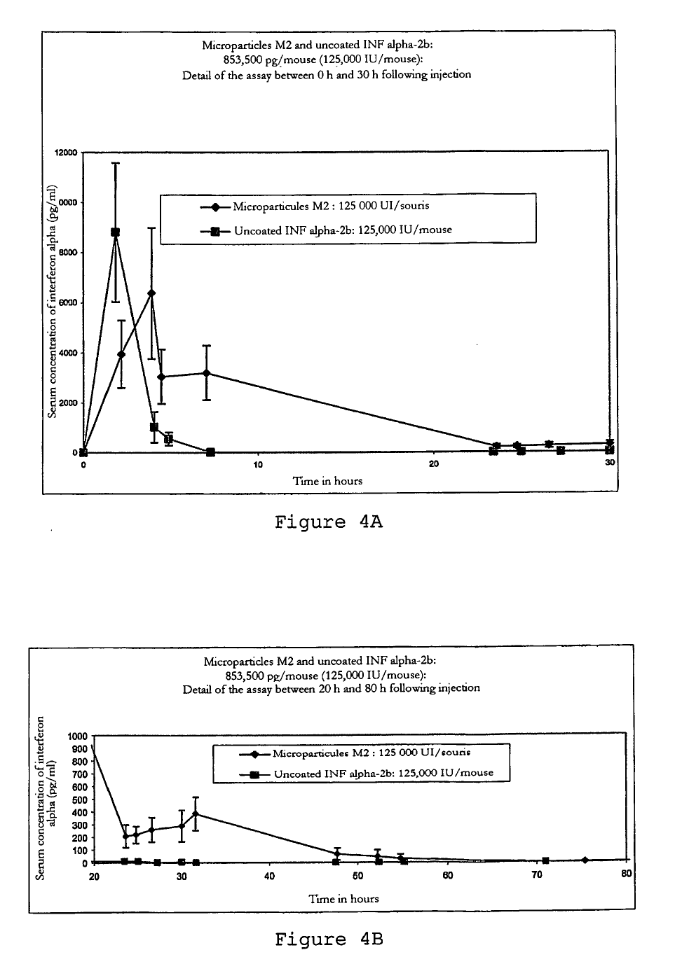

FIG. 4 represents the serum concentration of interferon α in the mouse for a dose of interferon α-2b of 125,000 IU/mouse: (A) 0 h to 30 h after the injection of uncoated INFα-2b or of microparticles M2 prepared according to example 2 and suspended in the medium from example 5, (B) 20 h to 80 h after the injection of uncoated INFα-2b or of microparticles M2 prepared according to example 2 and suspended in the medium from example 5.

### EXAMPLE 1

### Preparation of Lipid Microparticles M1 Containing Interferon α-2b

The microparticles are prepared from a lyophilizate of interferon α-2b (INF®, Gautier Cassara, Argentina) and from a mixture of glycerides (Precirol® ATO 5, Gattefossé, France) that are solid at ambient temperature. The melting point of Precirol® ATO 5 is measured by differential scanning enthalpic analysis and is found equal to 56.2° C. Before their coating by Precirol® ATO 5, the particles of INF® protein lyophilizate containing interferon α-2b (INFα-2b) are crushed using a mortar, then sifted using a 25 μm screen. The sifted fraction smaller than 25 μm is used for the preparation of the microparticles. The mean diameter of the crushed and sifted protein lyophilizate particles, determined by laser granulometry, is approximately 15 μm. The INFα-2b content of the protein particles to be coated is measured using an enzyme-linked immunosorbent assay (ELISA) kit specific for human INFα-2b (R&D Systems). It is measured and found equal to 2.866×10⁶ pg of INFα-2b per mg of particles to be coated.

157.45 mg of protein lyophilizate particles to be coated and 3 g of Precirol® ATO 5 are introduced into a stainless steel reactor with an internal volume of 1 l. The reactor has a maximum operating pressure of 30 MPa and is equipped with a double wall allowing the control of temperature by the circulation of coolant. The reactor's cover is equipped with a magnetically-driven pendular stirring device. After introduction of the particles to be coated and the Precirol® ATO 5, the reactor is closed and carbon dioxide is introduced into the reactor up to a pressure of 9.2 MPa. During this operation in which carbon dioxide is introduced, temperature is maintained at 23° C. The medium is then stirred at a speed of 210 rpm. Then the temperature of the reactor is gradually increased to 45° C., which leads to an increase in the pressure to 20 MPa. Under these temperature and pressure conditions, the carbon dioxide is in a supercritical state and the Precirol® ATO 5 is solubilized in the carbon dioxide. The reactor thus contains a dispersion of the particles to be coated in a solution of Precirol® ATO 5. After 1 hour of stirring at 45° C. and 20 MPa, the temperature of the reactor is decreased gradually and in a controlled way to 17° C. During this cooling, the pressure decreases to approximately 6.5 MPa. The duration of the cooling step is 30 min. The decrease in temperature and pressure of the medium causes a phase separation of the solution of Precirol® ATO 5 in the carbon dioxide. The Precirol® ATO 5 settles on the surface of the particles to be coated and a granulation phenomenon occurs, which results in microparticles containing protein particles dispersed in the matrix of the Precirol® ATO 5. The pressure of the reactor is then decreased to atmospheric pressure by controlled release of the carbon dioxide through a vent line. The duration of this decompression stage is 45 minutes.

Upon return to atmospheric pressure, the reactor is opened and 2.45 g of coated microparticles are recovered. The microparticles are then sifted using a 150 μm screen. The screening yield is 91%. The screened microparticles, herein designated M1, are then packaged in glass bottles under nitrogen atmosphere.

The microparticles are observed using an optical microscope equipped with a heating stage, which makes it possible to heat the microparticles to a temperature of approximately 70° C. and thus to cause the melting of the Precirol® ATO 5. This observation demonstrates that microparticles M1 consist of protein particles dispersed in the Precirol® ATO 5 (FIG. 1).

The granulometric distribution of microparticles M1, determined using a laser granulometer equipped with a liquid measuring cell, is presented in table 1. The mean diameter of the particles is 101.5 μm.

TABLE 1
Granulometric distribution of microparticles M1
Microparticles M1

| | | |
|---|---|---|
| 10% | 35.24 | μm |
| 20% | 57.4 | μm |
| 50% | 97.8 | μm |
| 80% | 142.01 | μm |
| 90% | 171.15 | μm |

The microparticles M1 are analyzed by differential scanning enthalpic analysis. The microparticles M1 are subjected to an increase in temperature from 10° C. to 80° C. at a rate of 10° C./min. This thermal analysis shows that the melting peak of the microparticles M1 is 57.9° C., with a melting onset temperature measured at 50.7° C. No endothermic or exothermic phenomenon is measured for temperatures lower than 50.7° C.

The load factor (LF) of the microparticles M1 is measured by an immunoenzymatic assay (ELISA) using a kit specific for human INFα-2b (R&D Systems, item 41100-1). The load factor of the batch of microparticles M1 is 4.1% of protein lyophilizate, which is a titer of 146,487 pg of INFα-2b per mg of microparticles.

### EXAMPLE 2

### Preparation of Lipid Microparticles M2 Containing Interferon α-2b

A preparation protocol identical to that described in example 1 is used, with the exception that the quantity of INFα-2b protein lyophilizate particles to be coated is 77.24 mg for 3 g of Precirol® ATO 5 introduced into the 1 l reactor.

After coating the protein particles according to a procedure identical to that used in example 1, 2.42 g of coated microparticles are recovered. These microparticles are filtered using a 150 μm screen. The screening yield is 96.2%. The screened microparticles, herein designated M2, are then packaged in glass bottles under nitrogen atmosphere.

The microparticles M2 are observed using an optical microscope after melting of the Precirol® ATO 5. It is observed that the microparticles M2 consist of protein lyophilizate particles dispersed in the Precirol® ATO 5.

The granulometric distribution of microparticles M2, determined using a laser granulometer equipped with a liquid measuring cell, is presented in table 2. The mean diameter of microparticles M2 is 110.5 μm.

TABLE 2
Granulometric distribution of microparticles M2
Microparticles M2

| | | |
|---|---|---|
| 10% | 32.38 | μm |
| 20% | 60.01 | μm |
| 50% | 105.6 | μm |
| 80% | 157.55 | μm |
| 90% | 191.9 | μm |

The microparticles M2 are analyzed by differential scanning enthalpic analysis. The microparticles M2 are subjected to an increase in temperature from 10° C. to 80° C. at a rate of 10° C./min. This thermal analysis shows that the melting peak of the microparticles M2 is 60° C., with a melting onset temperature measured at 48.3° C. No endothermic or exothermic phenomenon is measured for temperatures lower than 48.3° C.

The load factor of the microparticles M2 is determined by an immunoenzymatic assay (ELISA) using a kit specific for human INFα-2b (R&D Systems). The load factor (LF) of the batch of microparticles M2 is 1.6% of protein lyophilizate, which is a titer of 45,893 pg of INFα-2b per mg of microparticles.

### EXAMPLE 3

### The Use of Microparticles M1 to Improve the Bioavailability of Interferon α-2b (INFα-2b) in a Subcutaneous Injection

### 3.1. Procedure.

### 3.1.1. Preparation of Dispersions of Interferon α-2b Microparticles.

The batch of lipid microparticles M1 containing interferon α-2b (INFα-2b) coated with Precirol® ATO 5, obtained by following the protocol described in example 1, is used.

Two solutions which make it possible to disperse these microparticles before injection into the animal are prepared as follows:

1) Surfactant solution: in a 250 ml beaker, precisely weigh:
5 g of Lutrol® F68 (BASF),
5 g of Solutol® HS15 (BASF),
0.15 g of Montanox® 20PHA (SEPPIC),
8.33 g of D-Mannitol (CARLO ERBA).

100 ml of water for injection (WFI) is added using a test-tube. Stirring using a magnetic bar is maintained until the constituents are completely solubilized and a limpid solution is obtained. Sterile filtration of this solution is carried out using a 0.2 μm PES filter (Nalgene syringe filter).
Carboxymethyl cellulose (CMC) solution: in a 250 ml beaker:
Precisely pour 100 ml of WFI measured using a test-tube,
Weigh exactly 1.25 g of CMC (Blanose 7LF, Hercules),
Add the CMC all at once to the 100 ml of WFI under stirring by a magnetic bar.

Stirring is maintained until complete dissolution. Sterile filtration of this solution is carried out using a 0.2 μm PES filter (Nalgene syringe filter). Seven weight measurements of 263 mg ±1.5% are carried out and labeled microparticles M1 A to G in 20 ml type-I glass bottles equipped with a bromobutyl stopper that can be crimped.

For each bottle the precise volume X of total reconstitution solution that must be added to obtain a final interferon α-2b concentration of 3,793,333 pg/ml is calculated. Into the bottle containing the weighed microparticles is added a volume of surfactant solution equal to 60% of the volume X calculated previously.

The bottle is closed with a bromobutyl stopper, without crimping, and left under mixing for 5 minutes at a speed of 22 rpm. After 5 minutes, mixing is stopped, the bottle is recovered and a volume of CMC solution equal to 40% of the volume X calculated previously is added. The bottle is crimped and again mixed for 5 minutes at a speed of 22 rpm. The bottle is then placed in a Vortex Top-Mix 94323 mixer (Heidolph) for 2 seconds at maximum continuous mixing.

### 3.1.2. Preparation of the Uncoated Interferon α-2b Solution.

A solution containing uncoated Gautier Cassara interferon α-2b is prepared in an identical way to that of the redispersion solution: 91.7 mg ±1.5% of interferon α-2b is added to a 100 ml beaker in order to obtain a final interferon concentration of 3,793,333 pg/ml. The solution obtained is distributed into 7 20 ml type-I glass bottles equipped with a bromobutyl stopper that can be crimped.

### 3.1.3. Treatment of Animals and Sampling.

The total number of male Swiss mice, of 8 to 10 weeks in age, having received a subcutaneous injection of microparticles or of uncoated interferon alpha-2b is 70. Each mouse is numbered (universal coding by ear marks) from 1 to 70, and 7 batches of 10 mice are formed (batches A to G).

Each microparticle or uncoated interferon formulation study was analyzed for one week. The injections began in the morning after 9:00. Injection of the 10 mice constituting batch A was carried out first. Injection of the 10 mice constituting batch B was carried out next, then batch C, and so on, through batch G. The exact time injection began for each batch is noted, which makes it possible to correct the results according to the exact, not theoretical, time separating the injection from the sampling for each batch.

Aspiration of the microparticle suspension into the syringes was carried out with a 20 gauge needle (1 needle per batch), and the injections were carried out with 25 gauge needles (needle changed for each mouse). The solutions were homogenized on a Vortex mixer for 2 seconds between each sampling.

| Day | Number of hours after injection | Batch sampled |
|---|---|---|
| Tuesday | 2 | A |
| ″ | 4 | B |
| ″ | 5 | C |
| Wednesday | 8 | D |
| ″ | 24 | E |
| ″ | 26 | F |
| ″ | 28 | G |
| ″ | 30 | A |
| ″ | 32 | B |
| Thursday | 48 | C |
| ″ | 53 | D |
| ″ | 56 | E |
| Friday | 72 | F |
| ″ | 77 | G |
| Monday | 144 | A |
| ″ | 149 | B |

The 10 mice constituting the same batch were numbered, and the samples were taken in the same order as the injections, so that the time separating the injection and the sampling was the same for all the mice of the same batch. The exact time that sampling of each batch began was noted. The batches of mice of the same analysis were subject to alternated sampling over the course of the week, so that no batch of mice underwent more than one sampling per day, according to the following protocol: the sampling was stopped once the ELISA assays revealed an insignificant concentration of circulating interferon α-2b (the limit of quantification of the ELISA test was 12.5 IU/ml).

The volume of blood taken from the retro-orbital sinus of each mouse was 400 μl. The blood samples were centrifuged and the sera taken and frozen immediately, until needed for the ELISA assays. The assay of the interferon α-2b serum concentration of each sample collected was carried out using the ELISA technique (immunoenzymatic assay). For certain samples presenting a concentration of circulating interferon α-2b beyond the range of the ELISA assay, a dilution was carried out with PBS buffer, pH 7.4, 0.01% Montanox 20 DF, 0.02% sodium azide.

### 3.2. Results.

All of the serum results obtained with microparticles M1 are summarized in table 3 below:

TABLE 3
Serum concentration of human interferon α-2b as a function of
time following the injection of a suspension of microparticles
M1 at a concentration of 500,000 IU per mouse.

| | | Concentration (pg/ml) | |
|---|---|---|---|
| Theoretical time | Real time (h) | Mean | Standard deviation |
| 0 | 0.00 | 0 | 0 |
| 2 | 2.75 | 21051.76 | 6059.95 |
| 4 | 4.50 | 10571.51 | 2565.49 |
| 5 | 5.17 | 8141.79 | 1099.58 |
| 8 | 7.42 | 5701.16 | 1240.16 |
| 25 | 23.50 | 298.34 | 97.14 |
| 27 | 25.08 | 223.47 | 71.09 |
| 29 | 27.00 | 247.99 | 54.49 |
| 31 | 30.50 | 257.40 | 54.53 |
| 33 | 32.08 | 281.83 | 103.99 |
| 48 | 48.00 | 121.37 | 44.90 |
| 53 | 52.42 | 115.75 | 37.85 |
| 56 | 55.08 | 71.51 | 31.53 |
| 72 | 71.00 | 61.99 | 28.96 |
| 77 | 75.42 | 39.24 | 15.04 |
| 144 | 144.58 | 0.00 | 0.00 |
| 149 | 149.25 | 0.00 | 0.00 |

All of the serum results obtained with uncoated Gautier Cassara (INF®) interferon α-2b (INFα-2b) are summarized in table 4 below:

TABLE 4
Serum concentration of human interferon α-2b as a function of
time following the injection of a solution of uncoated
interferon α-2b (INF ®) at a concentration of
500,000 IU per mouse.

| | | Concentration (pg/ml) | |
|---|---|---|---|
| Theoretical time | Real time (h) | Mean | Standard deviation |
| 0 | 0.00 | 0 | 0 |
| 2 | 2.00 | 33737.17 | 5188.49 |
| 4 | 3.75 | 13749.07 | 3595.19 |
| 5 | 4.67 | 4118.72 | 830.51 |
| 8 | 7.33 | 482.33 | 115.71 |
| 25 | 23.33 | 149.19 | 32.23 |
| 27 | 25.08 | 100.44 | 18.73 |
| 29 | 26.92 | 125.74 | 34.78 |
| 31 | 30.00 | 83.13 | 25.04 |
| 33 | 31.67 | 77.90 | 20.24 |
| 48 | 47.67 | 19.00 | 12.66 |
| 53 | 52.42 | 33.33 | 7.54 |
| 56 | 55.25 | 15.43 | 5.25 |
| 72 | 71.42 | 7.19 | 5.50 |
| 77 | 75.92 | 6.34 | 4.37 |

The concentrations of interferon alpha assayed over the course of time in the serum of the animals having received either uncoated interferon α-2b or microparticles M1 are compared in FIGS. 1A to 1E.

The area under the curve (AUC) representative of the absolute bioavailability of the protein, calculated by triangular and rectangular modeling of the areas obtained between each sampling time, is presented in the following tables 5 and 6, in which the concentrations are expressed in IU/ml and not in pg/ml. (1 IU=6.828 pg of Gautier Cassara interferon α-2b).

TABLE 5
Calculation of the area under the curve (AUC): Uncoated
interferon α-2b (in (IU/ml) * h)

| Time | Concentration | Triangular area | Rectangular area | Total area |
|---|---|---|---|---|
| 0 | 0.000 | / | / | / |
| 2 | 4941.003 | 4941.0 | / | 4941.0 |
| 3.75 | 2013.630 | 2561.5 | 3523.9 | 6085.3 |
| 4.67 | 603.211 | 648.8 | 555.0 | 1203.7 |
| 7.33 | 70.641 | 708.3 | 187.9 | 896.2 |
| 23.33 | 21.850 | 390.3 | 349.6 | 739.9 |
| 25.08 | 14.711 | 6.2 | 25.7 | 32.0 |
| 26.92 | 18.415 | 3.4 | 27.1 | 30.5 |
| 30.00 | 12.174 | 9.6 | 37.5 | 47.1 |
| 31.67 | 11.408 | 0.6 | 19.1 | 19.7 |
| 47.67 | 2.783 | 69.0 | 44.5 | 113.5 |
| 52.42 | 4.882 | 5.0 | 13.2 | 18.2 |
| 55.25 | 2.260 | 3.7 | 6.4 | 10.1 |
| 71.42 | 1.053 | 9.8 | 17.0 | 26.8 |
| 75.92 | 0.93 | 0.3 | 4.2 | 4.5 |
| | | | Total: | 14168.5 |

TABLE 6
Calculation of the area under the curve: Microparticles M1

| Time | Concentration | Triangular area | Rectangular area | Total area |
|---|---|---|---|---|
| 0 | 0.000 | / | / | / |
| 2.75 | 3083.151 | 4239.3 | / | 4239.3 |
| 4.5 | 1548.259 | 1343.0 | 2709.5 | 4052.5 |
| 5.17 | 1192.413 | 119.2 | 798.9 | 918.1 |
| 7.42 | 834.968 | 402.1 | 1878.7 | 2280.8 |
| 23.50 | 43.693 | 6361.8 | 702.6 | 7064.4 |
| 25.08 | 32.729 | 8.7 | 51.7 | 60.4 |
| 27.00 | 36.319 | 3.4 | 62.8 | 66.3 |
| 30.50 | 37.698 | 2.4 | 127.1 | 129.5 |
| 32.08 | 41.276 | 2.8 | 59.6 | 62.4 |
| 48.00 | 17.775 | 187.1 | 283.0 | 470.1 |
| 52.42 | 16.952 | 1.8 | 74.9 | 76.7 |
| 55.08 | 10.474 | 8.6 | 27.9 | 36.5 |
| 71.00 | 9.080 | 11.1 | 144.6 | 155.6 |
| 75.42 | 5.740 | 7.4 | 25.4 | 32.8 |
| | | | Total: | 19645.4 |

The area under the curve (AUC) obtained for the lipid microparticles M1 containing the protein lyophilizate is 38.6% higher than the AUC obtained for the protein administered alone (uncoated) at the same dose.

### EXAMPLE 4

### The Use of the Microparticles M2 to Improve the Bioavailability of Interferon α-2b (IFNα-2b) in a Subcutaneous Injection

### 4.1. Procedure.

### 4.1.1. Preparation of Dispersions of Microparticles.

The batch of lipid microparticles M2 containing interferon α-2b (IFNα-2b) coated with Precirol® ATO 5, obtained by following the protocol described in example 2, is used.

The two solutions which make it possible to disperse these microparticles before injection into the animal are prepared in an identical way to that of example 3. Seven weight measurements of 204 mg ±2% are carried out and labeled microparticles M2 A to G in 20 ml type-I glass bottles equipped with a bromobutyl stopper that can be crimped. For each bottle the precise volume X of total reconstitution solution that must be added to obtain a final interferon concentration of 948,333 pg/ml is calculated.

Into the bottle containing the weighed microparticles is added a volume of surfactant solution equal to 60% of the volume X calculated previously.

The bottle is closed with a bromobutyl stopper, without crimping, and left under mixing for 5 minutes at a speed of 22 rpm. After 5 minutes, mixing is stopped, the bottle is recovered and a volume of CMC solution equal to 40% of the volume X calculated previously is added. The bottle is crimped and again mixed for 5 minutes at a speed of 22 rpm. The bottle is then placed in a Vortex Top-Mix 94323 mixer (Heidolph) for 2 seconds at maximum continuous mixing.

### 4.1.2. Preparation of the Uncoated of Interferon α-2b Solution.

A solution containing uncoated Gautier Cassara interferon α-2b is prepared in an identical way: 22.9 mg ±1.5% of interferon α-2b is added to a 100 ml beaker in order to obtain a final interferon α-2b concentration of 948,333 pg/ml. The solution obtained is distributed into 7 20 ml type-I glass bottles equipped with a bromobutyl stopper that can be crimped.

### 4.1.3. Treatment of Animals and Sampling.

The total number of male Swiss mice, of 8 to 10 weeks in age, having received a subcutaneous injection of microparticles or of uncoated interferon alpha-2b is 70. Each mouse is numbered (universal coding by ear marks) from 1 to 70, and 7 batches of 10 mice are formed (batches A to G).

Each microparticle or uncoated interferon formulation study was analyzed for one week. The injections and the sampling were carried out following a protocol identical to that of example 3.

The batches of mice of the same analysis were subject to alternated sampling over the course of the week, so that no batch of mice underwent more than one sampling per day, according to the following protocol:

| Day | Number of hours after injection | Batch sampled |
|---|---|---|
| Monday | 2 | A |
| ″ | 4 | B |
| ″ | 5 | C |
| ″ | 8 | D |
| Tuesday | 24 | E |
| ″ | 26 | F |
| ″ | 28 | G |
| ″ | 30 | A |
| ″ | 32 | B |
| Wednesday | 48 | C |
| ″ | 53 | D |
| ″ | 56 | E |
| Thursday | 72 | F |
| ″ | 77 | G |
| Friday | 96 | A |
| ″ | 101 | B |

The sampling was stopped once the ELISA assays revealed an insignificant concentration of circulating interferon alpha (the limit of quantification of the ELISA test was 12.5 IU/ml). The volume of blood taken from the retro-orbital sinus of each mouse was 400 μl. The blood samples were centrifuged and the sera taken and frozen immediately, until needed for the ELISA assays.

The assay of the human interferon α-2b serum concentration of each sample collected was carried out using the ELISA technique (immunoenzymatic assay). For certain samples presenting a concentration of circulating interferon α-2b beyond the range of the ELISA assay, a dilution was carried out with PBS buffer, pH 7.4, 0.01% Montanox 20 DF, 0.02% sodium azide.

### 4.2. Results.

All of the serum results obtained with microparticles M2 are summarized in table 7 below:

TABLE 7
Serum concentration of human interferon α-2b in animals as a
function of time following the injection of a suspension of
microparticles M2 at a concentration of 125,000 IU per mouse.

| | | Concentration (pg/ml) | |
|---|---|---|---|
| Theoretical time | Time in hours | Mean | Standard deviation |
| 0 | 0.00 | 0 | 0 |
| 2 | 2.58 | 10194.39 | 3921.13 |
| 4 | 4.00 | 8183.94 | 2930.55 |
| 5 | 4.92 | 5557.55 | 3788.00 |
| 8 | 7.50 | 3606.26 | 1596.34 |
| 25 | 23.08 | 271.62 | 84.12 |
| 27 | 24.92 | 324.22 | 149.05 |
| 29 | 26.75 | 247.36 | 109.86 |
| 31 | 30.25 | 245.73 | 41.64 |
| 33 | 31.92 | 243.42 | 52.81 |
| 48 | 48.17 | 90.57 | 41.18 |
| 53 | 52.58 | 129.82 | 59.76 |
| 56 | 54.42 | 98.86 | 56.60 |
| 72 | 70.92 | 9.82 | 9.98 |
| 77 | 75.67 | 4.22 | 7.88 |
| 96 | 96.25 | 0.00 | 0.00 |
| 101 | 100.75 | 0.00 | 0.00 |

All of the serum results obtained with the uncoated interferon α-2b are summarized in table 8 below:

TABLE 8
Serum concentration of human interferon α-2b in animals as a
function of time following the injection of a solution of
uncoated interferon alpha at a concentration of 125,000 IU per
mouse.

| | | Concentration (pg/ml) | |
|---|---|---|---|
| Theoretical time | Time in hours | Mean | Standard deviation |
| 0 | 0.00 | 0 | 0 |
| 2 | 1.83 | 8812.16 | 2776.03 |
| 4 | 4.08 | 1022.84 | 618.94 |
| 5 | 4.92 | 548.68 | 266.12 |
| 8 | 7.33 | 15.89 | 21.03 |
| 25 | 23.42 | 9.17 | 13.70 |
| 27 | 25 | 9.40 | 13.47 |
| 29 | 27.25 | 0.75 | 1.25 |
| 31 | 30 | 5.76 | 7.31 |
| 33 | 31.66 | 0.00 | 0.00 |
| 48 | 47.5 | 0.00 | 0.00 |
| 53 | 52.33 | 0.00 | 0.00 |
| 56 | 55.17 | 0.00 | 0.00 |
| 72 | 71 | 0.00 | 0.00 |

The concentrations in interferon alpha assayed over the course of time in the serum of animals having received either interferon α-2b or microparticles M2 are compared in FIGS. 2A and 2B.

The area under the curve is calculated by the method described in example 3 and is presented in following tables 9 and 10:

TABLE 9
Calculation of the area under the curve (AUC): Uncoated
interferon α-2b (in (IU/ml) * h)

| Time | Concentration | Triangular area | Rectangular area | Total area |
|---|---|---|---|---|
| 0 | 0.0 | / | / | / |
| 1.83 | 1290.6 | 1180.9 | / | 1180.9 |
| 4.08 | 149.8 | 1283.4 | 337.1 | 1620.4 |
| 4.92 | 80.4 | 29.2 | 67.5 | 96.7 |
| 7.33 | 2.3 | 94.0 | 5.6 | 99.6 |
| 23.42 | 1.3 | 7.9 | 21.6 | 29.5 |
| 25 | 1.4 | 0.0 | 2.1 | 2.1 |
| 27.25 | 0.1 | 1.4 | 0.2 | 1.7 |
| 30 | 0.8 | 1.0 | 0.3 | 1.3 |
| 31.66 | 0.0 | 0.7 | 0.0 | 0.7 |
| 47.5 | 0.0 | 0.0 | 0.0 | 0.0 |
| 52.33 | 0.0 | 0.0 | 0.0 | 0.0 |
| 55.17 | 0.0 | 0.0 | 0.0 | 0.0 |
| 71 | 0.0 | 0.0 | 0.0 | 0.0 |
| | | | Total: | 3033.0 |

TABLE 10
Calculation of the area under the curve (AUC): Microparticles
M2 (in (IU/ml) * h)

| Time | Concentration | Triangular area | Rectangular area | Total area |
|---|---|---|---|---|
| 0 | 0.00 | / | / | / |
| 2.58 | 1493.03 | 1926.0 | 0.0 | 1926.0 |
| 4.00 | 1198.59 | 209.1 | 1702.0 | 1911.0 |
| 4.92 | 813.94 | 176.9 | 748.8 | 925.8 |
| 7.50 | 528.16 | 368.7 | 1362.6 | 1731.3 |
| 23.08 | 39.78 | 3804.5 | 619.8 | 4424.2 |
| 24.92 | 47.48 | 7.1 | 73.2 | 80.3 |
| 26.75 | 36.23 | 10.3 | 66.3 | 76.6 |
| 30.25 | 35.99 | 0.4 | 126.0 | 126.4 |
| 31.92 | 35.65 | 0.3 | 59.5 | 59.8 |
| 48.17 | 13.26 | 181.9 | 215.5 | 397.4 |
| 52.58 | 19.01 | 12.7 | 58.5 | 71.2 |
| 54.42 | 14.48 | 4.2 | 26.6 | 30.8 |
| 70.92 | 1.44 | 107.6 | 23.7 | 131.3 |
| 75.67 | 0.62 | 1.9 | 2.9 | 4.9 |
| 96.25 | 0.00 | 6.4 | 0.0 | 6.4 |
| 100.75 | 0.00 | 0.0 | 0.0 | 0.0 |
| | | | Total: | 11903.4 |

The area under the curve (AUC) obtained for the lipid microparticles M2 containing the protein lyophilizate is higher by a factor of 3.9 than the AUC obtained for the protein administered alone (uncoated) at the same dose.

### EXAMPLE 5

### The Use of the Lipid Microparticles M2 with a Reconstitution Solution Containing Lutrol® F127 to Improve the Bioavailability of Interferon α-2b (INFα-2b) in a Subcutaneous Injection

### 5.1. Procedure.

### 5.1.1. Preparation of the Dispersion of Microparticles.

The same batch of lipid microparticles M2 of interferon α-2b (IFNα-2b) coated with Precirol® ATO 5 as that of example 4, whose load factor (LF) is 1.6% protein lyophilizate, is used.

A solution which make it possible to disperse these microparticles before injection into the animal is prepared in the following manner: in a 250 ml beaker, precisely weigh:
3 g of Lutrol® F68 (BASF),
3 g of Solutol® HS15 (BASF),
0.09 g of Montanox® 20PHA (SEPPIC),
5.1 g of D-Mannitol (CARLO ERBA).

100 ml of water for injection (WFI) is added using a test-tube, and stirring using a magnetic bar is maintained until the constituents are completely solubilized and a limpid solution is obtained. The beaker is placed in an ice-water bath. 7.5 g of Lutrol® F127 (BASF) is weighed and it is added all at once into the preceding solution. Stirring is maintained until complete dissolution. Sterile filtration of this solution is carried out using a 0.2 μm PES filter (Nalgene syringe filter).

Seven weight measurements of 204 mg ±2% are carried out and labeled microparticles M2 A to G in 20 ml type-I glass bottles equipped with a bromobutyl stopper that can be crimped. For each bottle the precise volume X of total reconstitution solution that must be added to obtain a final interferon concentration of 948,333 pg/ml is calculated. This volume of solution is added into the bottle containing the weighed microparticles M2. The bottle is crimped with a bromobutyl stopper and is left to mix for 10 min at a speed of 22 rpm. The bottle is then placed in a Vortex Top-Mix 94323 mixer (Heidolph) for 2 seconds at maximum continuous mixing.

The results obtained with the uncoated interferon α-2b as described in example 4 are used for purposes of comparison.

### 5.1.2. Treatment of Animals and Sampling.

The total number of male Swiss mice, of 8 to 10 weeks in age, having received a subcutaneous injection of microparticles or of uncoated interferon alpha-2b is 70. Each mouse is numbered (universal coding by ear marks) from 1 to 70, and 7 batches of 10 mice are formed (batches A to G).

Each microparticle or uncoated interferon formulation study was analyzed for one week. The injections and the sampling was carried out following a protocol identical to that of example 4. The batches of mice of the same analysis were subject to alternated sampling over the course of the week, so that no batch of mice underwent more than one sampling per day, according to the following protocol:

| Day | Number of hours after injection | Batch sampled |
|---|---|---|
| Monday | 2 | A |
| ″ | 4 | B |
| ″ | 5 | C |
| ″ | 8 | D |
| Tuesday | 24 | E |
| ″ | 26 | F |
| ″ | 28 | G |
| ″ | 30 | A |
| ″ | 32 | B |
| Wednesday | 48 | C |
| ″ | 53 | D |
| ″ | 56 | E |
| Thursday | 72 | F |
| ″ | 77 | G |
| Friday | 96 | A |
| ″ | 101 | B |

The sampling was stopped once the ELISA assays revealed an insignificant concentration of circulating interferon α-2b (the limit of quantification of the ELISA test was 12.5 IU/ml). The volume of blood taken from the retro-orbital sinus of each mouse was 400 μl. The blood samples were centrifuged and the sera taken and frozen immediately, until needed for the ELISA assays.

The human interferon alpha-2b serum concentration of each sample collected was measured using the ELISA technique (immunoenzymatic assay). For certain samples presenting a concentration of circulating interferon α-2b beyond the range of the ELISA assay, a dilution was carried out with PBS buffer, pH 7.4, 0.01% Montanox 20 DF, 0.02% sodium azide.

### 5.2. Results.

All of the serum results obtained with microparticles M2 in the reconstitution solution containing Lutrol® F127 are summarized in table 11 below:

TABLE 11
Serum concentration of human interferon α-2b as a function of
time following the injection of a suspension of microparticles
M2 at a concentration of 125,000 IU per mouse.

| | | Concentration (pg/ml) | |
|---|---|---|---|
| Theoretical time | Time in hours | Mean | Standard deviation |
| 0 | 0.00 | 0 | 0 |
| 2 | 2.17 | 3945.54 | 1347.67 |
| 4 | 3.92 | 6366.02 | 2613.22 |
| 5 | 4.50 | 3044.54 | 1085.99 |
| 8 | 7.08 | 3202.59 | 1087.07 |
| 25 | 23.58 | 207.89 | 90.07 |
| 27 | 24.75 | 218.50 | 66.70 |
| 29 | 26.58 | 259.17 | 96.96 |
| 31 | 30.00 | 289.06 | 124.85 |
| 33 | 31.58 | 384.13 | 130.94 |
| 48 | 47.67 | 65.37 | 46.70 |
| 53 | 52.17 | 46.15 | 51.63 |
| 56 | 54.75 | 28.74 | 32.74 |
| 72 | 70.92 | 0.00 | 0.00 |
| 77 | 75.42 | 1.92 | 5.76 |
| 96 | 96.25 | 3.42 | 7.95 |

The concentrations in interferon alpha assayed over the course of time in the serum of animals having received either interferon α-2b or microparticles M2 taken up in the reconstitution solution containing Lutrol® F127 are compared in FIGS. 3A and 3B.

The area under the curve is calculated by the method described in example 3 and is presented in following tables 12 and 13, in which the concentrations are expressed in IU/ml and not in pg/ml. Recall that 1 IU=6.828 pg of Gautier Cassara interferon α-2b.

TABLE 12
Calculation of the area under the curve (AUC): Uncoated
interferon α-2b (in (IU/ml) * h)

| Time | Concentration | Triangular area | Rectangular area | Total area |
|---|---|---|---|---|
| 0 | 0.0 | / | / | / |
| 1.83 | 1290.6 | 1180.9 | / | 1180.9 |
| 4.08 | 149.8 | 1283.4 | 337.1 | 1620.4 |
| 4.92 | 80.4 | 29.2 | 67.5 | 96.7 |
| 7.33 | 2.3 | 94.0 | 5.6 | 99.6 |
| 23.42 | 1.3 | 7.9 | 21.6 | 29.5 |
| 25 | 1.4 | 0.0 | 2.1 | 2.1 |
| 27.25 | 0.1 | 1.4 | 0.2 | 1.7 |
| 30 | 0.8 | 1.0 | 0.3 | 1.3 |
| 31.66 | 0.0 | 0.7 | 0.0 | 0.7 |
| 47.5 | 0.0 | 0.0 | 0.0 | 0.0 |
| 52.33 | 0.0 | 0.0 | 0.0 | 0.0 |
| 55.17 | 0.0 | 0.0 | 0.0 | 0.0 |
| 71 | 0.0 | 0.0 | 0.0 | 0.0 |
| | | | Total: | 3033.0 |

TABLE 13
Calculation of the area under the curve for Microparticles M2;
new suspension solution (in (IU/ml) * h)

| Time | Concentration | Triangular area | Rectangular area | Total area |
|---|---|---|---|---|
| 0 | 0.0 | / | / | / |
| 2.17 | 577.8 | 627.0 | / | 627.0 |
| 3.92 | 932.3 | 310.2 | 1011.2 | 1321.4 |
| 4.50 | 445.9 | 141.1 | 258.6 | 399.7 |
| 7.08 | 469.0 | 29.9 | 1150.4 | 1180.3 |
| 23.58 | 30.4 | 3618.4 | 502.4 | 4120.8 |
| 24.75 | 32.0 | 0.9 | 35.6 | 36.5 |
| 26.58 | 38.0 | 5.5 | 58.6 | 64.0 |
| 30.00 | 42.3 | 7.5 | 129.8 | 137.3 |
| 31.58 | 56.3 | 11.0 | 66.9 | 77.9 |
| 47.67 | 9.6 | 375.6 | 154.0 | 529.6 |
| 52.17 | 6.8 | 6.3 | 30.4 | 36.7 |
| 54.75 | 4.2 | 3.3 | 10.9 | 14.1 |
| 70.92 | 0.0 | 34.0 | 0.0 | 34.0 |
| 75.42 | 0.3 | 0.6 | 0.0 | 0.6 |
| 96.25 | 0.5 | 2.3 | 5.9 | 8.2 |
| | | | Total: | 8588.1 |

The area under the curve (AUC) obtained for the lipid microparticles M2 containing the protein lyophilizate, redispersed in a solution of Lutrol® F127 is higher by a factor of 2.83 than the AUC obtained for the protein administered alone (uncoated) at the same dose.

Table 14 summarizes the values of the parameter AUC, representative of the absolute bioavailability of the protein, found in examples 3, 4 and 5.

TABLE 14

| | | Dose | AUC |
|---|---|---|---|
| Formulation | LF(%) | (IU/mouse) | ((IU/ml) * h) |
| Interferon alpha 2-b in | / | 500,000 | 14169 |
| solution (reference) | | | |
| Microparticles M1 | 4.1 | 500,000 | 19645 |
| redispersed in CMC | | | |
| solution | | | |
| Interferon alpha 2-b in | / | 125,000 | 3033 |
| solution (reference) | | | |
| Microparticles M2 | 1.6 | 125,000 | 11903 |
| redispersed in CMC | | | |
| solution | | | |
| Microparticles M2 | 1.6 | 125,000 | 8588 |
| redispersed in Lutrol F127 | | | |
| solution | | | |

1. A method of increasing the bioavailability of a protein active ingredient in a patient in need thereof, comprising administering to the patient via subcutaneous or intramuscular injection an effective amount of glycerol dipalmitostearate and the protein active ingredient, whereby the bioavailability of the protein active ingredient is increased.

2. The method of claim 1, wherein the glycerol dipalmitostearate is in type I atomized form.

3. The method of claim 1, wherein the protein active ingredient is a peptide or a peptide derivative selected from the group consisting of somatotropin analogs, somatomedin-C, gonadotropin-releasing hormone, follicle-stimulating hormone, luteinizing hormone-releasing hormone (LHRH), analogs of LHRH, leuprolide, nafarelin, goserelin, LHRH agonists, LHRH antagonists, growth hormone releasing factor, calcitonin, colchicine, gonadotropins, chorionic gonadotropin, oxytocin, octreotide, somatotropin, amino-acid associated somatotropin, vasopressin, adrenocorticotropic hormone, epidermal growth factor, prolactin, somatostatin, protein-associated somatropin, cosyntropin, lypressin, thyrotropin-releasing hormone, secretin, pancreozymin, enkephalin, glucagon, and endocrine agents secreted internally and distributed by blood flow.

4. The method of claim 1, wherein the protein active ingredient is a protein selected from the group consisting of alpha-1 antitrypsin, factor VIII, factor IX, coagulation factors, insulin, peptide hormones, growth hormone, cortical androgen-stimulating hormone, thyroid stimulating hormone, pituitary hormones, parathyroid hormone-related protein, interferons alpha, interferon beta, interferon gamma, interferon delta, erythropoietin (EPO), growth factors, granulocyte colony-stimulating factor (GCSF), granulocyte-macrophage colony-stimulating factor (GMCSF), nerve growth factor (NGF), neurotrophic factors, GDNF, insulin-like growth factors, tissue plasminogen activator, CD4, dDAVP, tumor necrosis factor (TNF), pancreatic enzymes, lactase, cytokines, interleukin-1 receptor antagonists, interleukin-2, tumor necrosis factor (TNF) receptor, tumor suppressing proteins, and cytotoxic proteins.

5. The method of claim 4, wherein the protein active ingredient is selected from the group consisting of cytokines and interferons.

6. The method of claim 5, wherein the protein active ingredient is interferon α-2b.

7. The method of claim 1, wherein the protein active ingredient is contained in particles ranging between 0.1 μm and 100 μm in size.

8. The method of claim 1, wherein the protein active ingredient is formulated with at least one lipids in the form of a nanocapsule, microcapsule, nanosphere, microsphere, cylindrical implant, or discoidal implants.

9. The method of claim 1, wherein the protein active ingredient is contained in a formulated system having a size in the range between 1 μm and 500 μm.

10. The method of claim 9, wherein the protein active ingredient is contained in particles that are formulated in a lipid particulate system having a diameter in the range between 10 μm and 100 μm.

11. The method of claim 9, wherein the protein active ingredient is contained in particles that are formulated in a lipid implant of 10 μm to 300 μm in diameter.

12. The method of claim 1, wherein the protein active ingredient is contained in particles that have a protein active ingredient content between 0.01% and 100%.

13. The method of claim 12, wherein the protein active ingredient is contained in particles that have a protein active ingredient content between 0.1% and 95%.

14. The method of claim 1, wherein the protein active ingredient is contained in a formulated system comprising matrix of microspheres that are prepared starting from glycerol dipalmitostearate in type I atomized form and that contain 0.5% to 10% of particles of the protein active ingredient.

15. The method of claim 14, wherein the matrix of microspheres are prepared by encapsulation of the protein active ingredient using a fluid at supercritical pressure.

16. The method of claim 1, wherein the protein active ingredient is interferon α-2b.

17. A microsphere matrix comprising a protein active ingredient, wherein said microsphere matrix is prepared starting from glycerol dipalmitostearate in type I atomized form and contains from 0.5% to 10% of particles of the protein active ingredient.

18. A method of preparing a formulated system that increases the bioavailability of a protein active ingredient, comprising preparing the formulated system starting from the protein active ingredient and at least one lipid soluble in supercritical CO₂, wherein the formulated system increases the bioavailability of the protein active ingredient in a patient in need thereof when the formulated system is administered to the patient via subcutaneous or intramuscular injection.

19. The method of claim 18, wherein the at least one lipid is solid at ambient temperature.

20. The method of claim 7, wherein the protein active ingredient is contained in particles ranging between 1 μm and 25 μm in size.

21. The method of claim 8, wherein the protein active ingredient is formulated with at least one lipid in the form of a matrix structure.

22. The method of claim 13, wherein the protein active ingredient is contained in particles that have a protein active ingredient content between 0.5% and 25%.

23. The method of claim 22, wherein the protein active ingredient is contained in particles that have a protein active ingredient content between 1% and 10%.

24. The method of claim 14, wherein the protein active ingredient is contained in a formulated system comprising matrix of microspheres that are prepared starting from glycerol dipalmitostearate in type I atomized form and that contain 1% to 5% of particles of the protein active ingredient.

25. The microsphere matrix of claim 17, wherein the microsphere matrix contains from 1% to 5% of particles of the protein active ingredient.

26. The microsphere matrix of claim 17, wherein the protein active ingredient is interferon α-2b.
